(19) United States
(12) Patent Application Publication
(10) Pub. No.: US 2004/0210287 A1
(43) Pub. Date: Oct. 21, 2004

(54) Portable cooling or heating device for applying cryotherapy

(76) Inventor: Judy L. Greene, Columbus, OH (US)

Correspondence Address: JAMES RAY & ASSOCIATES, 2640 PITCAIRN ROAD, MONROEVILLE, PA, 15146, US

(21) Appl. No.: 10/419,608

(22) Filed: Apr. 21, 2003

(51) Int. Cl. A61F 7/00

(52) U.S. Cl. 607/112; 607/114

(57) ABSTRACT

An apparatus used to apply cryotherapy comprising at least one gel pack of a predetermined size and predetermined shape, a retaining means of a predetermined size and predetermined shape which is engageable with at least a portion of a gel pack and at least a portion of a body part for retaining the gel pack in any desired orientation while being disposed on the body part. It also includes at least one variable temperature means that is engageable with at least a portion of the gel pack for sustaining a predetermined temperature the gel pack during a predetermined time period.

### FIELD OF INVENTION

The present invention relates, in general, to devices that are worn on the contour of a person's body for the purpose of facilitating healing. More particularly, this invention relates to a device that conforms to any structural contour of a person's body and retains a lightweight portable cooling and heating device for maintaining a desired temperature.

### BACKGROUND OF THE INVENTION

Cryotherapy, the application of ice or other cold materials, is proven in the initial treatment and rehabilitation of a variety of muscular-skeletal injuries, including sprains, strains, contusions, and fractures. It has also been found to be useful in the rehabilitation process after surgery.

Generally, the goals of cryotherapy are to limit the formation of edema, to reduce pain, and to reduce muscle spasms. There are various types of devices, systems, and methods used today to apply cryotherapy including ice massage, ice bags, cold towels, cold gel packs, cold chemical packs, and vapor coolant sprays. However, none of these maintain a constant temperature for an extended period of time.

It is known that clinical response to cryotherapy is improved if the cooling means, such as an ice bag or gel pack, is cooled to a suitable low temperature and applied proximally to the body part. The clinical response to cryotherapy is also improved if the cooling means is kept sufficiently cold and applied continuously to the area during treatment. Generally, clinical response improves the longer the cooling means are applied to the area. Unfortunately, most cooling means generally lose their coldness in a relatively short time and must be replaced over an extended treatment. Also, applying the cold cooling means for a relatively long period to an area can cause frostbite in the adjacent skin and superficial tissues. Moreover, the initial application of the cooling means to the skin surface elicits a pain response which, after continuous applications, give way to an aching or throbbing sensation and then eventually to numbness.

The device and system used in the application of cryotherapy can also affect the clinical response. It is known, for example that for optimal treatment, the cooling means should be positioned on opposite sides of the body part so that deep, even penetration of cold treatment may be given. Also, since extended cryotherapy treatment requires the continuous application of a sufficiently cold cooling means, several sets of sufficiently cold cooling means must be readily available for exchange during treatment. Many devices, such as those disclosed in U.S. Pat. No. 4,753,240, and 4,676,247, and 5,148,804 fail to address the problems listed above.

### SUMMARY OF THE INVENTION

The present invention provides an apparatus used to apply cryotherapy comprising at least one gel pack of a predetermined size and predetermined shape, a retaining means of a predetermined size and predetermined shape which is engageable with at least a portion of a gel pack and at least a portion of a body part for retaining the gel pack in any desired orientation while being disposed on the body part. It also includes at least one variable temperature means that is engageable with at least a portion of the gel pack for sustaining a predetermined temperature the gel pack during a predetermined time period.

### OBJECTS OF THE INVENTION

It is, therefore, one of the primary objects of the present invention to provide a portable cooling or heating device for applying cryotherapy that maintains a substantially constant temperature for an indefinite period of time.

It is also an object of the present invention to provide a portable cooling or heating device for applying cryotherapy that is reusable.

Another object of the present invention is to provide a portable cooling or heating device for applying cryotherapy that has a variable temperature range.

Another object of the present invention is to provide a portable cooling or heating device for applying cryotherapy that is adjusted by user to fit any structural contour of a person's body.

Still another object of the present invention is to provide a portable cooling or heating device for applying cryotherapy that limits bruising and swelling.

Yet another object of the present invention is to provide a portable cooling or heating device for applying cryotherapy having a retaining means which provides a predetermined resistance for limiting the expansion of at least one gel pack, and for maintaining a body contour.

These and various other objects and advantages of this invention will become more readily apparent to those persons skilled in the art after a full reading of the following detailed description, particularly, when such description is read in conjunction with the attached drawings as described below and the appended claims.

### BRIEF DESCRIPTION OF THE DRAWINGS

FIG. 1 is a detailed view of a retaining means.

FIG. 2 is one embodiment of gel pack.

FIG. 3 is an electrical diagram of an electronic circuit.

FIG. 4 is an exploded view of an electronic circuit assembly

### BRIEF DESCRIPTION OF THE PRESENTLY PREFERRED AND ALTERNATE EMBODIMENTS OF THE INVENTION

Prior to proceeding with the more detailed description of the present invention it should be noted that, for the sake of clarity, identical components, which have identical functions have been designated by identical reference numerals throughout the drawing Figures.

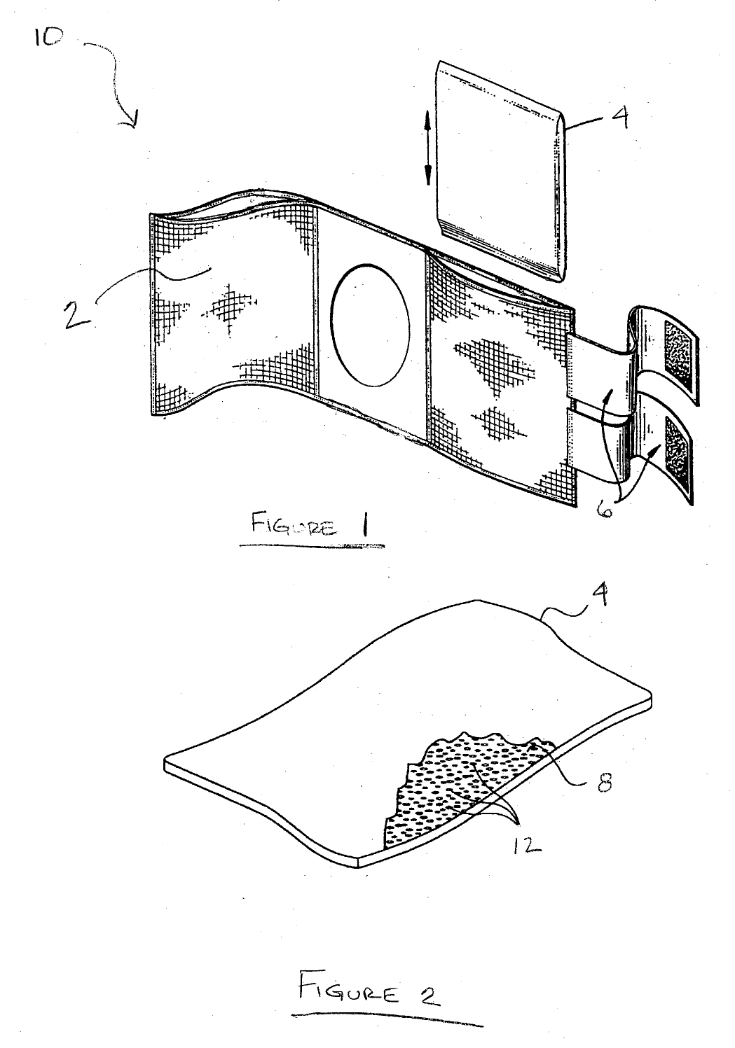

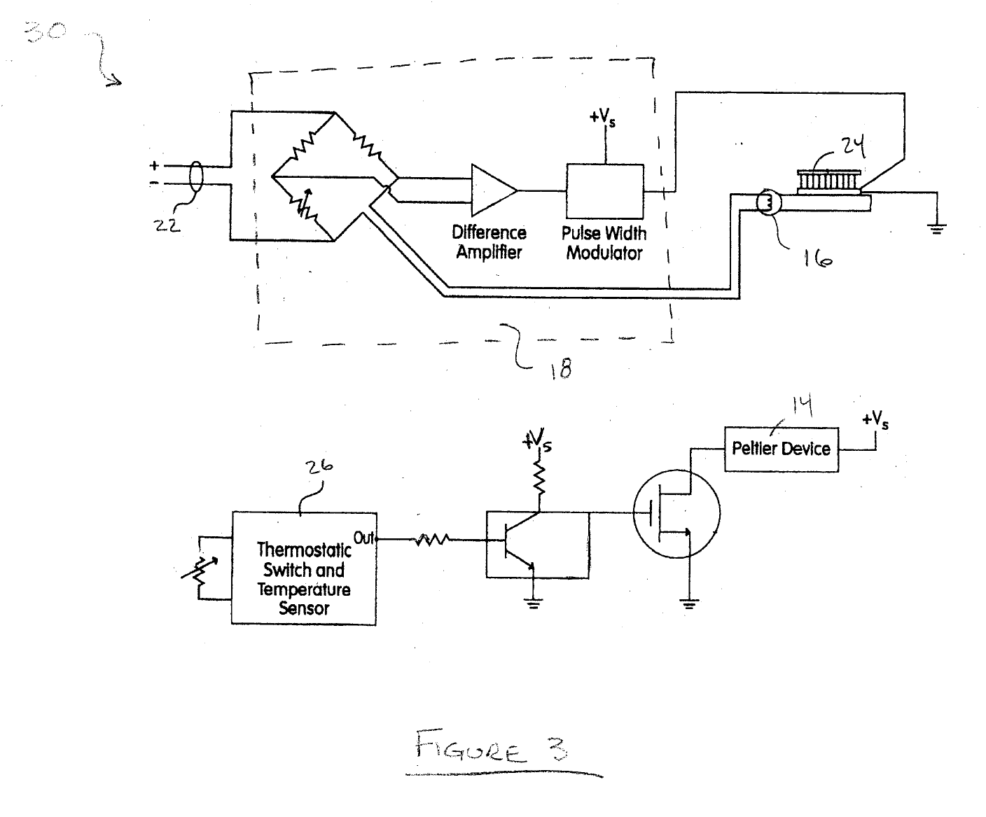

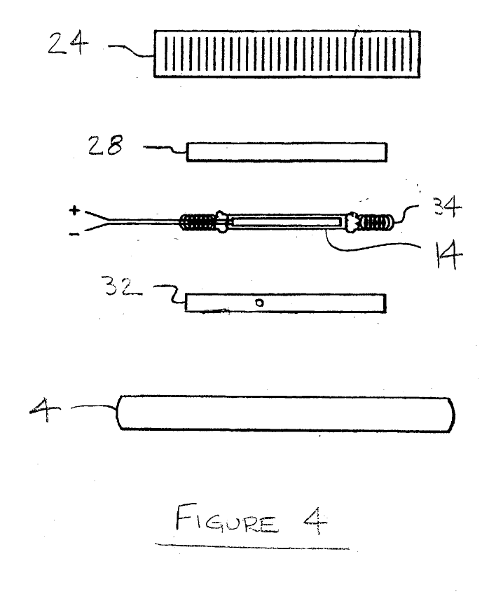

Reference is now made to FIGS. 1 through 3. Illustrated therein is a presently preferred embodiment of an apparatus, generally designated 10, used to apply cryotherapy. In the presently preferred embodiment, the apparatus 10 used to apply cryotherapy comprises at least one gel pack 4 of a predetermined size and predetermined shape. The gel pack 4 is from a family of gel packs having a flexible shape and containing a filler material 8 mixed with a high thermal conductivity heat transfer material 12. The heat transfer material 12 is one of either flakes or pellets and is relatively inert in said filler material 8. The apparatus 10 also includes a retaining means 2 of a predetermined size and predetermined shape, made from a flexible material, engageable with at least a portion of a gel pack 4 and at least a predetermined portion of a body part for retaining the gel pack 4 in any desired orientation while being disposed on a body part. Additionally, the retaining means 2 is capable of securing itself to a body part through the use of a retention means 6. The presently preferred embodiment also includes at least one variable temperature means 30 engageable with at least a portion of a gel pack 4 for sustaining a predetermined temperature of the gel pack 4 during a predetermined time period. The variable temperature means 30 is an electronic circuit assembly 30 which also includes a source of power 22. The source of power 22 can be either an alternating current or direct current source, such as either a standard wall outlet or battery.

Referring now to FIG. 4, the electronic circuit assembly 30 also includes at least one predetermined solid-state device 14 having a warm side and a cool side connected to a source of power 22. The warm side is attached to a first plate 28 and the cool side is attached to a second plate 32. Additionally there is an insulating means 34 engaged with these plates 28 and 32 to discourage thermal transformation between them. The plates, 28 and 32, are made from a high thermal conductivity material such as copper. There is a radiator means 24, such as a heatsink, affixed to the first plate 28 for providing air flow and for encouraging the transfer of heat energy from the radiator means 24 to ambient air. The solid-state device 14 is from a family of solid-state devices commonly known as thermoelectric modules, more specifically it is a Peltier Device. It also includes a control circuit 18 which is connected to the solid-state device 14 and to the source of power 22 and is used for controlling the solid-state device 14. There is a feed back means 16 connected to the control circuit 18 and the source of power 22 used for reporting feedback signals to the control circuit 18 for improving the control of the solid-state device 14. The control circuit 18 includes a thermostatic switch and temperature sensor 26 for selecting a predetermined temperature setting of the solid-state device 14, and to sense the temperature of gel pack 4.

While both the presently preferred and a number of alternative embodiments of the present invention have been described in detail above it should be understood that various other adaptations and modifications of the present invention can be envisioned by those persons who are skilled in the relevant art without departing from either the spirit of the invention or the scope of the appended claims.

I claim:

1. An apparatus used to apply cryotherapy, said apparatus comprising:
a) at least one gel pack of a predetermined size and predetermined shape;
b) a retaining means of a predetermined size and predetermined shape engageable with at least a portion of said at least one gel pack and at least a predetermined portion of a body part for retaining said at least one gel pack in any desired orientation while being disposed on said predetermined portion of such body part; and
c) at least one variable temperature means engageable with at least a portion of said at least one gel pack for sustaining a predetermined temperature of said at least one gel pack during a predetermined time period.

2. An apparatus used to apply cryotherapy, according to claim 1, wherein said gel pack is from a family of gel packs having a flexible shape and containing a filler material mixed with a high thermal conductivity heat transfer material.

3. An apparatus used to apply cryotherapy, according to claim 2, herein said heat transfer material is at least one of flakes and pellets and is relatively inert in said filler material.

4. An apparatus used to apply cryotherapy, according to claim 1, wherein said retaining means is made from a predetermined material.

5. An apparatus used to apply cryotherapy, according to claim 4, wherein said predetermined material is flexible.

6. An apparatus used to apply cryotherapy, according to claim 1, wherein said retaining means further includes a retention means for securing said retaining means on such body part.

7. An apparatus used to apply cryotherapy, according to claim 1, wherein said at least one variable temperature means is an electronic circuit assembly and said apparatus further includes a source of power connected to said electronic circuit assembly.

8. An apparatus used to apply cryotherapy, according to claim 7, wherein said electronic circuit assembly further includes:
a) at least one predetermined solid-state device having a warm side and a cool side connected to said source of power;
b) a control circuit connected to said predetermined solid-state device and to said source of power for controlling said predetermined solid-state device;
c) a feed back means connected to said control circuit and to said source of power for reporting feedback signals to said control circuit for improving control of said predetermined solid-state device;
d) a first plate attached to said warm side of said predetermined solid-state device;
e) a second plated attached to said cool side of said predetermined solid-state device;
f) an insulating means engageable with said first plate and said second plate for discouraging thermal transformation between said first plate and said second plate; and
g) a radiator means affixed to said first plate for providing air flow and for encouraging the transfer of heat energy from said radiator means to ambient air.

9. An apparatus used to apply cryotherapy, according to claim 8, herein said predetermined solid-state device is from a family of solid-state devices commonly known as thermoelectric modules.

10. An apparatus used to apply cryotherapy, according to claim 9, wherein said predetermined solid-state device is a Peltier Device.

11. An apparatus used to apply cryotherapy, according to claim 8, wherein said first plate and said second plate are manufactured from a high thermal conductivity material.

12. An apparatus used to apply cryotherapy, according to claim 8, wherein said high thermal conductivity material is copper.

13. An apparatus used to apply cryotherapy, according to claim 8, wherein said radiator means is a heat sink.

14. An apparatus used to apply cryotherapy, according to claim 8, wherein said control circuit further includes a thermostat for selecting a predetermined temperature setting of said predetermined solid-state device.

15. An apparatus used to apply cryotherapy, according to claim 14, wherein said thermostat further includes a temperature sensor means.

16. An apparatus used to apply cryotherapy, according to claim 15, wherein said temperature sensor means senses a temperature of said at least one gel pack.

17. An apparatus used to apply cryotherapy, according to claim 8, wherein said source of power is at least one of an alternating current and a direct current source.

18. An apparatus used to apply cryotherapy, according to claim 8, wherein said direct current source is a battery.
